(19) United States
(12) Patent Application Publication
(10) Pub. No.: US 2026/0224394 A1
(43) Pub. Date: Aug. 6, 2026

(54) ATTACHMENT TOOL AND PRESSURE SENSE PRESENTATION APPARATUS

(71) Applicant: Sony Interactive Entertainment Inc., Tokyo (JP)

(72) Inventors: Yuriho Higuchi, Kanagawa (JP); Yuichi Machida, Kanagawa (JP); Toru Kuronuma, Kanagawa (JP); Hitoshi Nakamura, Tokyo (JP)

(21) Appl. No.: 19/578,643

(22) Filed: Mar. 25, 2026

Related U.S. Application Data
(63) Continuation of application No. PCT/JP2023/034993, filed on Sep. 26, 2023.

(51) Int. Cl. A61F 7/08; A61H 23/02

(52) U.S. Cl. CPC A61F 7/08; A61H 23/02

(57) ABSTRACT

An attachment tool includes a strip that has a first surface and a second surface on a side opposite to the first surface and extends in a first direction, a through-hole that is provided to an end of the strip positioned in a direction opposite to the first direction and penetrates the strip, a slit that is provided to the strip in the first direction with respect to the through-hole, extends along the first direction, and penetrates the strip, and a locking section that is provided to one surface of the first surface and the second surface, contacts the other surface of the first surface and the second surface, and locks the other surface.

### CROSS-REFERENCE TO RELATED APPLICATIONS

This application is a Continuation of International Application No. PCT/JP2023/034993, having a filing date of September 26, 2023. This disclosure of the prior application is considered part of the disclosure of this application

The present specification relates to an attachment tool and a pressure sense presentation apparatus.

### BACKGROUND

Heretofore, there has been known a tactile device for imparting tactile information to a user

### SUMMARY

Another tactile device is a glove-shaped device mounted on a hand of the user and incorporates a shape-memory wire. The shape-memory wire is provided at a position in the tactile device that corresponds to each finger of the hand and also at a position corresponding to the back of the hand. When an electric current passes through the shape-memory wire, it heats the shape-memory wire and thereby changes the shape thereof. The change in the shape of the shape-memory wire applies a pressure to the joints of the fingers and the palm of the hand of the user. Therefore, the tactile device can give the user tactile information as if touching or gripping an object.

Another tactile device is a glove-shaped device mounted on a hand. The tactile device suffers a problem in that, since the size of a hand is different from individual to individual, it is necessary to prepare a tactile device that matches the size of a hand of the user in order for the user to use it.

Consequently, there has been a demand for an attachment tool that can be attached to an attachment target portion regardless of the fact that the attachment target portion is different from individual to individual.

An attachment tool according to a first aspect of the present specification includes a strip that has a first surface and a second surface on a side opposite to the first surface and extends in a first direction, a through-hole that is provided to an end of the strip positioned in a direction opposite to the first direction and penetrates the strip, a slit that is provided to the strip in the first direction with respect to the through-hole, extends along the first direction, and penetrates the strip, and a locking section that is provided to one surface of the first surface and the second surface, contacts the other surface of the first surface and the second surface, and locks the other surface.

A pressure sense presentation apparatus according to a second aspect of the present specification includes the attachment tool according to the first aspect and a pressing section that is provided to the strip and presses an attachment target portion to which the attachment tool is attached.

### BRIEF DESCRIPTION OF THE DRAWINGS

FIG. 1 is a plan view illustrating a pressure sense presentation apparatus according to a first implementation.

FIG. 2 is a plan view illustrating the pressure sense presentation apparatus according to the first implementation.

FIG. 3 is a view explanatory of a method of attaching an attachment tool according to the first implementation.

FIG. 4 is a view explanatory of the method of attaching the attachment tool according to the first implementation.

FIG. 5 is a view explanatory of the method of attaching the attachment tool according to the first implementation.

FIG. 6 is a view explanatory of the method of attaching the attachment tool according to the first implementation.

FIG. 7 is a schematic view illustrating a pressing apparatus according to the first implementation.

FIG. 8 is a schematic view illustrating an example of the layout of pressing sections according to the first implementation.

FIG. 9 is a schematic view illustrating an example of the layout of pressing sections according to the first implementation.

FIG. 10 is a schematic view illustrating an example of the layout of pressing sections according to the first implementation.

FIG. 11 is a schematic view illustrating an example of the layout of a pressing section according to the first implementation.

FIG. 12 is a schematic view illustrating an example of the layout of pressing sections according to the first implementation.

FIG. 13 is a schematic view illustrating an example of the layout of pressing sections according to the first implementation.

FIG. 14 is a schematic view illustrating an example of the layout of pressing sections according to the first implementation.

FIG. 15 is a schematic view illustrating a pressing apparatus and a temperature adjusting apparatus according to a second implementation.

FIG. 16 is a schematic view illustrating a pressing apparatus and a temperature adjusting apparatus according to a third implementation.

FIG. 17 is a schematic view illustrating another example of attaching the attachment tool.

### DETAILED DESCRIPTION

### First implementation

A first implementation of the present specification will be described below with reference to the drawings.

### Configuration of a pressure sense presentation apparatus

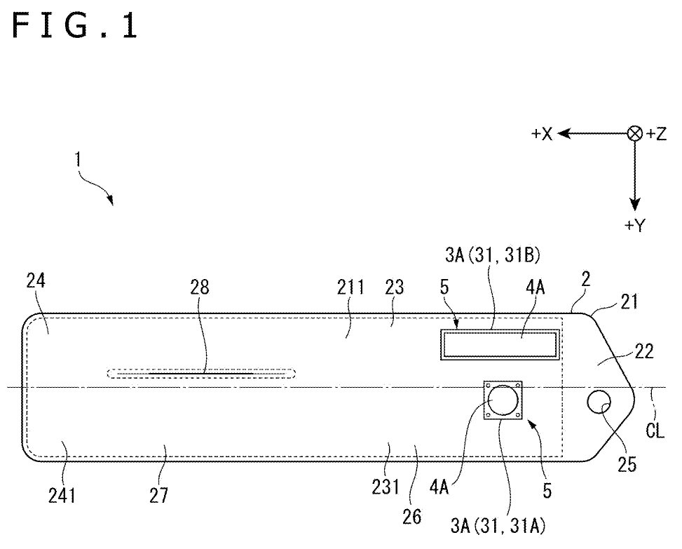

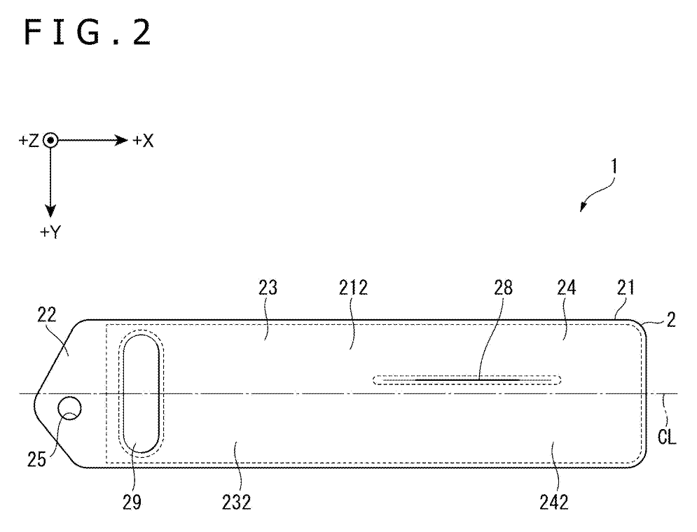

FIGS. 1 and 2 are plan views illustrating a pressure sense presentation apparatus 1 according to a first implementation. More specifically, FIG. 1 is a plan view illustrating the pressure sense presentation apparatus 1 as viewed from the side of a first surface 211 of a strip 21 of an attachment tool 2, and FIG. 2 is a plan view illustrating the pressure sense presentation apparatus 1 as viewed from the side of a second surface 212 of the strip 21.

The pressure sense presentation apparatus 1 according to the present implementation is mounted on part of the body of a user in use. The pressure sense presentation apparatus 1 presses the body of the user to impart a pressure sensation to the user. According to the present implementation, the pressure sense presentation apparatus 1 is mounted on a hand of the user and presses the hand.

As illustrated in FIGS. 1 and 2, the pressure sense presentation apparatus 1 includes the attachment tool 2, a pressing apparatus 3A, a temperature adjusting apparatus 4A, and a vibration generating apparatus 5.

The pressure sense presentation apparatus 1 according to the present implementation will be described below as mounted on the left hand of the user in use.

### Configuration of the attachment tool

The attachment tool 2 is mounted on part of the body described above. The attachment tool 2 illustrated in FIGS. 1 and 2 is an attachment tool for use on the left hand and is mounted on the left hand as an attachment target portion.

As illustrated in FIG. 1, the attachment tool 2 has the strip 21, a through-hole 25, a first arrangement section 26, a second arrangement section 27, and a slit 28. In addition, as illustrated in FIG. 2, the attachment tool 2 has a locking section 29.

### Configuration of the strip

The strip 21 extends along a first direction. The strip 21 is of a substantially rectangular shape that is elongate in the first direction. When the strip 21 is attached to the left hand, it has the first surface 211 facing the palm and back of the left hand and the second surface 212 on the side opposite to the first surface 211.

The strip 21 has a first portion 22, a second portion 23, and a third portion 24 that are interconnected. The first portion 22, the second portion 23, and the third portion 24 are arrayed along the first direction.

In the description below, three directions that are orthogonal to each other are referred to as a +X direction, a +Y direction, and a +Z direction. In FIG. 1, the leftward direction represents the +X direction, the downward direction represents the +Y direction, and the direction toward the sheet of FIG. 1 represents the +Z direction. According to the present implementation, the first direction is represented by the +X direction, a second direction is represented by the +Y direction, and the direction from the first surface 211 toward the second surface 212 is represented by the +Z direction. Although not illustrated, a direction opposite to the +X direction is referred to as a -X direction, a direction opposite to the +Y direction is referred to as a -Y direction, and a direction opposite to the +Z direction is referred to as a -Z direction. In FIG. 1, the -X direction is represented by the rightward direction, the -Y direction is represented by the upward direction, and the -Z direction is represented by the direction away from the sheet of FIG. 1.

The first portion 22 is provided on the end of the strip 21 in the -X direction. The first portion 22 is of a substantially pentagonal shape as viewed from the -Z direction and has an end in the -X direction that is defined by two sides of a triangular shape protruding in the -X direction. The through-hole 25 that penetrates the strip 21 in the +Z direction is provided in the first portion 22.

The through-hole 25 is of a circular shape. The through-hole 25 is spaced in the +Y direction from a centerline CL that crosses the middle on the strip 21 in the +Y direction and that is parallel to the +X direction. When the attachment tool 2 is attached to the left hand, the thumb of the left hand is inserted through the through-hole 25 in the +Z direction.

The expandability/contractibility of the first portion 22 described above is higher than the expandability/contractibility of the second portion 23 and the third portion 24. Therefore, when the attachment tool 2 is attached to the left hand, the first portion 22 is expanded or contracted.

The second portion 23 is provided between the first portion 22 and the third portion 24 in the +X direction.

The second portion 23 has a first surface 231 facing in the -Z direction and representing part of the first surface 211 of the strip 21. When the attachment tool 2 is attached to the left hand, the first surface 231 lies opposite to the palm of the left hand. The first arrangement section 26 that is of a substantially rectangular shape where pressing sections 31, to be described later, of the pressing apparatus 3A are placed is provided on the first surface 231.

The first arrangement section 26 is arranged such that as many pressing sections 31 as desired can be placed at desired positions on the first arrangement section 26. The pressing sections 31 placed on the first arrangement section 26 press the palm of the left hand. As described in detail later, the first arrangement section 26 engages with connecting sections 313 of the pressing sections 31, locking the pressing sections 31. The first arrangement section 26 described above may include a hook-and-loop fastener, for example.

As illustrated in FIG. 2, the second portion 23 has a second surface 232 facing the +Z direction and representing part of the second surface 212 of the strip 21. The locking section 29 is provided to the second surface 232.

The locking section 29 extends along the +Y direction on a portion of the second surface 232 near its end in the -X direction. Specifically, the locking section 29 extends along the +Y direction at a position on the side of the second surface 232 near the first portion 22. As described in detail later, when the attachment tool 2 is attached to the left hand, the locking section 29 contacts a first surface 241 of the third portion 24 that faces in the -Z direction, locking the third portion 24. The attachment tool 2 thus remains attached to the left hand. The locking section 29 described above may include a hook-and-loop fastener, for example.

As illustrated in FIGS. 1 and 2, the third portion 24 is largest among the first portion 22, the second portion 23, and the third portion 24. Specifically, the dimension of the third portion 24 in the +X direction is larger than the sum of the dimension of the first portion 22 in the +X direction and the dimension of the second portion 23 in the +X direction. Note that the dimension of the third portion 24 in the +Y direction is substantially the same as the dimension of each of the first portion 22 and the second portion 23 in the +Y direction.

The first surface 241 of the third portion 24 that faces in the -Z direction represents part of the first surface 211. When the attachment tool 2 is attached to the left hand, the first surface 241 is placed over the back and palm of the left hand. In other words, a region of the first surface 241 on its side near the second portion 23 lies opposite to the back of the left hand.

The third portion 24 has a second surface 242 facing in the +Z direction and represents part of the second surface 212.

The second arrangement section 27 and the slit 28 are provided on the third portion 24.

The second arrangement section 27 is provided substantially entirely over the first surface 241. As with the first arrangement section 26, the second arrangement section 27 is arranged such that as many pressing sections 31 as desired can be placed at desired positions on the second arrangement section 27. The pressing sections 31 placed on portions of the second arrangement section 27 that are opposite to the back of the left hand press the back of the left hand. Further, the second arrangement section 27 is arranged such that it can be locked by the locking section 29. The second arrangement section 27 described above may include a hook-and-loop fastener, for example.

The slit 28 is provided at a position spaced in the -Y direction from the centerline CL described above. The slit 28 extends along the +X direction and penetrates the third portion 24 along the +Z direction. As described in detail later, when the strip 21 with the thumb of the left hand inserted through the through-hole 25 is placed on the left hand over the palm and back thereof, the thumb of the left hand is inserted through the slit 28 in the +Z direction.

### Method of attaching the attachment tool

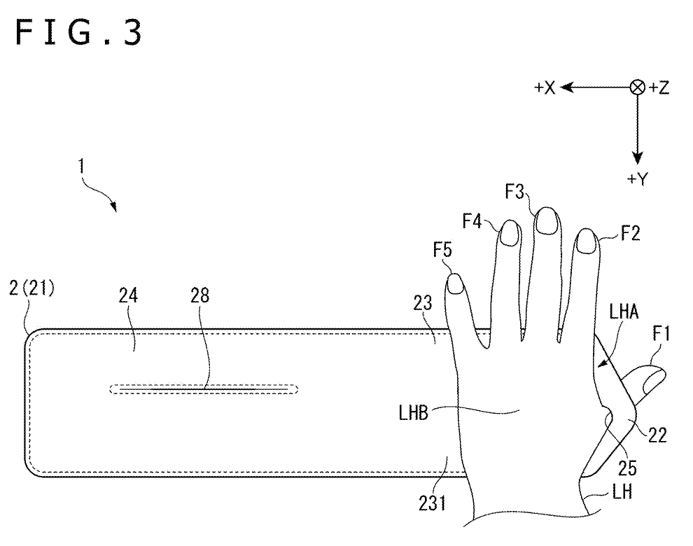

FIGS. 3 through 6 are views explanatory of a method of attaching the attachment tool 2 to a left hand LH.

When the attachment tool 2 is attached to the left hand LH, as illustrated in FIG. 3, a thumb F1 of the left hand LH is inserted through the through-hole 25 in the +Z direction, and the left hand LH is placed such that the palm of the left hand LH lies opposite to the first surface 231 of the second portion 23.

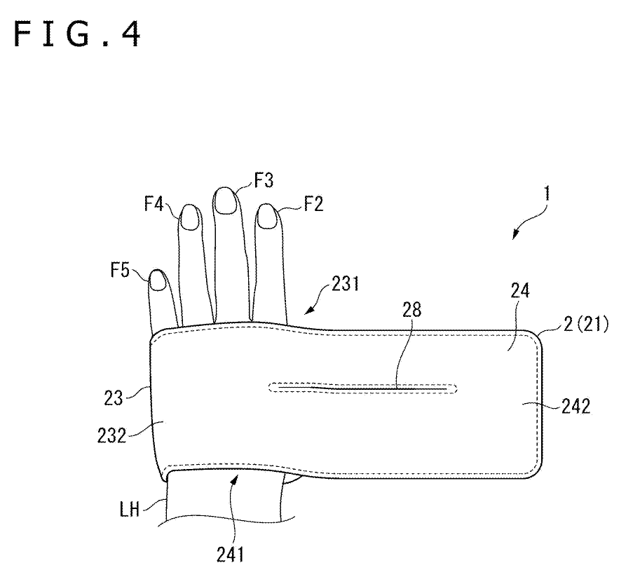

Then, as illustrated in FIG. 4, the strip 21 is wound around the left hand LH such that the first surface 231 of the second portion 23 lies along a palm LHA of the left hand LH and the first surface 241 of the third portion 24 lies along a back LHB of the left hand LH. The palm LHA of the left hand LH corresponds to a first surface of an attachment target portion, and the back LHB of the left hand LH corresponds to a second surface, opposite to the first surface, of the attachment target portion.

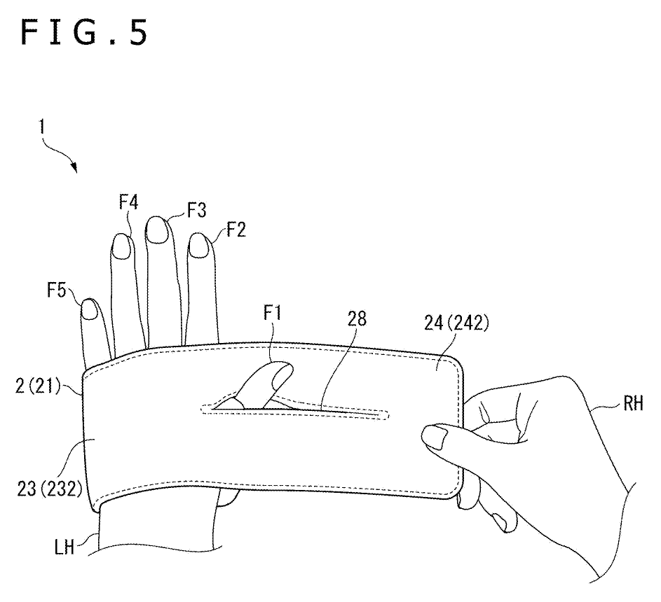

Thereafter, as illustrated in FIG. 5, the thumb F1 of the left hand LH is inserted through the slit 28 while a right hand RH, for example, is pulling the third portion 24 in the direction in which the strip 21 extends.

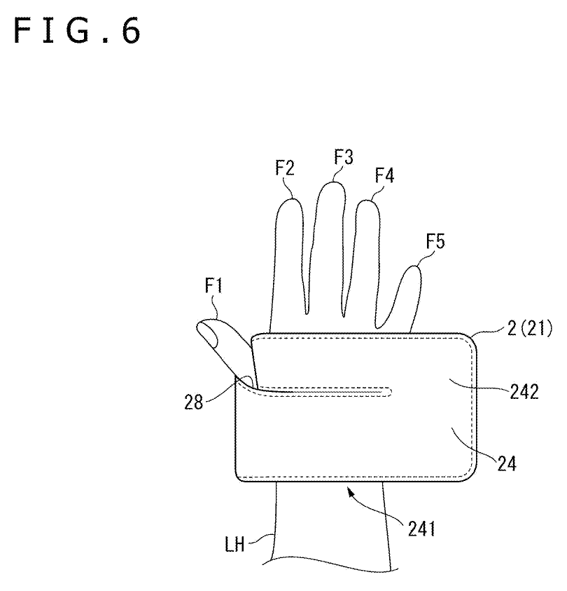

Then, the second arrangement section 27 (see FIG. 1) of the third portion 24 is brought into contact with the locking section 29 (see FIG. 2) provided to the second surface 232 of the second portion 23. The locking section 29 thus locks the second arrangement section 27 and hence the third portion 24, whereupon the attachment tool 2 is attached to the left hand LH, as illustrated in FIG. 6.

### Configuration of the pressing apparatus

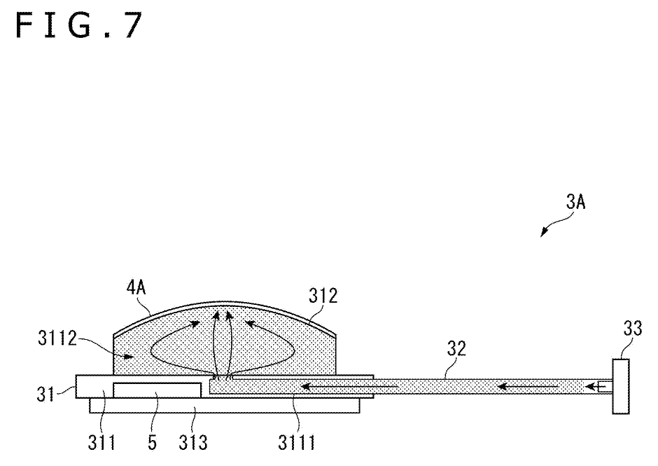

FIG. 7 is a schematic view illustrating the configuration of the pressing apparatus 3A.

The pressing apparatus 3A presses an attachment target portion of the user. As illustrated in FIG. 7, the pressing apparatus 3A has a pressing section 31, a tube 32, and a pump 33.

### Configuration of the pressing section

The pressing section 31 represents a section of the pressing apparatus 3A that presses the attachment target portion of the user. As illustrated in FIG. 1, the pressing section 31 is arranged such that it can be placed in the first arrangement section 26 and is also arranged such that it can be placed in the second arrangement section 27. As illustrated in FIG. 7, the pressing section 31 has a base 311, an expandable/contractible section 312, and a connecting section 313.

The base 311 is connected to the tube 32 and is arranged to allow fluid to flow therein. The base 311 has a fluid passage 3111 and an inlet chamber 3112.

Fluid is distributed from the tube 32 connected to the base 311 to the fluid passage 3111.

The inlet chamber 3112 is a space into which the fluid flows from the fluid passage 3111 and is covered with the expandable/contractible section 312.

The expandable/contractible section 312 presses the attachment target portion to which the attachment tool 2 is attached. According to the present implementation, the expandable/contractible section 312 presses the left hand LH as a target hand.

The expandable/contractible section 312 includes an expandable/contractible material such as silicone rubber sheet and is mounted on the base 311 in covering relation to the inlet chamber 3112. Further, the expandable/contractible section 312 is expanded by the fluid flowing into the inlet chamber 3112, pressing the attachment target portion that is opposite to the expandable/contractible section 312. Note that it is sufficient if the expandable/contractible section 312 is brought into contact with the attachment target portion when expanded, and the expandable/contractible section 312 may not necessarily be held in contact with the attachment target portion at all times.

The connecting section 313 refers to a section that connects the pressing section 31 to the attachment tool 2. Specifically, the connecting section 313 is provided to a surface of the base 311 remote from the expandable/contractible section 312 and mounts the base 311 on the first arrangement section 26 or the second arrangement section 27. Specifically, the connecting section 313 is provided to the surface of the base 311 that is opposite to the arrangement section 26 or 27 and is removable from the arrangement section 26 or 27. According to the present implementation, the connecting section 313 may include a hook-and-loop fastener that can be locked on the first arrangement section 26 or the second arrangement section 27.

### Configuration of the tube and the pump

The tube 32 is arranged such that the fluid can flow therethrough and is connected to the base 311 and the pump 33. The tube 32 introduces the fluid pumped from the pump 33 into the base 311.

The pump 33 pumps the fluid through the tube 32 to the pressing section 31. The fluid pumped from the pump 33 is distributed through the tube 32 to the fluid passage 3111 in the base 311 and flows through the fluid passage 3111 into the inlet chamber 3112. When the pressure in the inlet chamber 3112 rises, the expandable/contractible section 312 is expanded to press the attachment target portion. On the other hand, when the pump 33 stops pumping the fluid, the fluid is discharged from the inlet chamber 3112 by the contracting force of the expandable/contractible section 312. The expandable/contractible section 312 is contracted to release the attachment target portion from being pressed.

Note that the pump 33 may draw in the fluid from the inlet chamber 3112.

Further, the fluid pumped from the pump 33 may be either gas or liquid. If the pump 33 pumps gas, air around the pump 33 may be used as the gas. If the pump 33 pumps liquid, the pressing apparatus 3A may include a reservoir capable of holding the liquid to be pumped by the pump 33.

### Configuration of the temperature adjusting apparatus

The temperature adjusting apparatus 4A imparts at least one of a warm sensation and a cold sensation to the user. Specifically, the temperature adjusting apparatus 4A imparts at least one of a warm sensation and a cold sensation to the left hand LH as the attachment target portion. According to the present implementation, the temperature adjusting apparatus 4A includes a thermoelectric transducer such as a nichrome wire or a Peltier device mounted on an outer surface of the expandable/contractible section 312. If the temperature adjusting apparatus 4A includes a Peltier device, it imparts one of a warm sensation and a cold sensation to the attachment target portion, depending on the direction of a voltage and a current applied thereto. Specifically, the temperature adjusting apparatus 4A that includes a Peltier device is able to impart a warm sensation and a cold sensation by reversing the polarity of the Peltier device.

### Configuration of the vibration generating apparatus

The vibration generating apparatus 5 imparts a vibration to the user. According to the present implementation, the vibration generating apparatus 5 imparts a vibration through the pressing section 31 to the left hand LH. Specifically, the vibration generating apparatus 5 is provided on the base 311 and imparts a vibration to the left hand LH through the expandable/contractible section 312 that is contacting the left hand LH.

Note that the vibration generating apparatus 5 may be provided to the pressing section 31, e.g., the inlet chamber 3112. Alternatively, the vibration generating apparatus 5 may be provided on a constituent element other than the pressing section 31, e.g., the strip 21.

### Shape and size of the pressing section

The shape and size of the expandable/contractible section 312 of the pressing section 31 can be changed appropriately.

For example, of the two pressing sections 31 illustrated in FIG. 1, the pressing section denoted by 31A has an expandable/contractible section 312 that is of a circular shape as viewed from the -Z direction, and the pressing section denoted by 31B has an expandable/contractible section 312 that is of a rectangular as viewed from the -Z direction.

Therefore, the shape and size of the expandable/contractible section 312 and hence the shape and size of the pressing section 31 can be changed appropriately.

### Examples of the layout of pressing sections

In the example illustrated in FIG. 1, of the two pressing sections 31 provided to the first arrangement section 26 of the strip 21, the pressing section 31A is arranged at a position corresponding to a substantially central area of the palm of the left hand LH, and the pressing section 31B is arranged at a position on the palm of the left hand LH corresponding to the roots of an index finger F2, a middle finger F3, a ring finger F4, and a little finger F5 along the direction in which the fingers F2 through F5 are arrayed. In other words, the pressing section 31B is arranged at a position corresponding to the finger balls of the palm of the left hand LH.

However, the numbers of pressing sections 31 provided to the strip 21, the layout of the pressing sections 31, and the shape of the expandable/contractible section 312 are not necessarily limited to the above details.

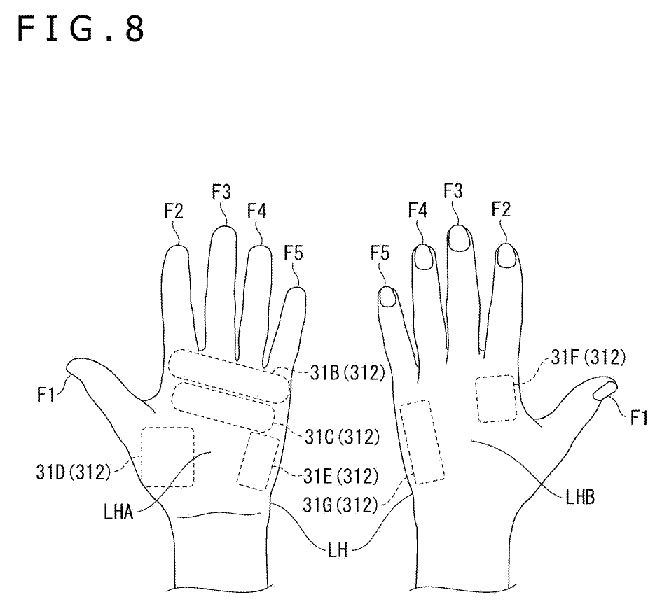

FIG. 8 is a schematic view illustrating an example of the layout of pressing sections 31 on the palm LHA and the back LHB of the left hand LH.

In the example of FIG. 8, four pressing sections 31 are provided to the first arrangement section 26 that is opposite to the palm LHA of the left hand LH, and two pressing sections 31 are provided to the second arrangement section 27 that is opposite to the back LHB of the left hand LH.

Each of the four pressing sections, denoted by 31B, 31C, 31D, and 31E, provided to the first arrangement section 26 has a rectangular expandable/contractible section 312. As is the case with the configuration illustrated in FIG. 1, the pressing section 31B is arranged at a position corresponding to the finger balls of the palm LHA. The expandable/contractible section 312 of the pressing section 31C is smaller in longitudinal dimension than the expandable/contractible section 312 of the pressing section 31B. The pressing section 31C is arranged parallel to the pressing section 31B at a position on the palm LHA that is closer to the wrist than the pressing section 31B.

The pressing section 31D is arranged at the root of the thumb F1 on the palm LHA, i.e., at a position corresponding to the ball of the thumb. The pressing section 31E is arranged at a position spaced toward the wrist from the root of the little finger F5, i.e., at a position corresponding to the ball of the little finger, on the palm LHA. The expandable/contractible section 312 of each of the pressing sections 31D and 31E is smaller in longitudinal dimension than the expandable/contractible section 312 of the pressing section 31C.

Each of the pressing sections 31F and 31G provided to the second arrangement section 27 has a rectangular expandable/contractible section 312. The pressing section 31F is provided at a position on the back LHB of the left hand LH corresponding to the root of the index finger F2. The pressing section 31G is provided at a position on the back LHB corresponding to a range from the root of the little finger F5 toward the wrist. The size of the expandable/contractible section 312 of the pressing section 31F is smaller than the size of the expandable/contractible section 312 of the pressing section 31E, and the size of the expandable/contractible section 312 of the pressing section 31G is larger than the size of the expandable/contractible section 312 of the pressing section 31E.

With the above layout of the pressing sections 31, when each of the pressing sections 31 presses the left hand LH, the pressure sense presentation apparatus 1 causes the user to feel a sensation of shaking hands.

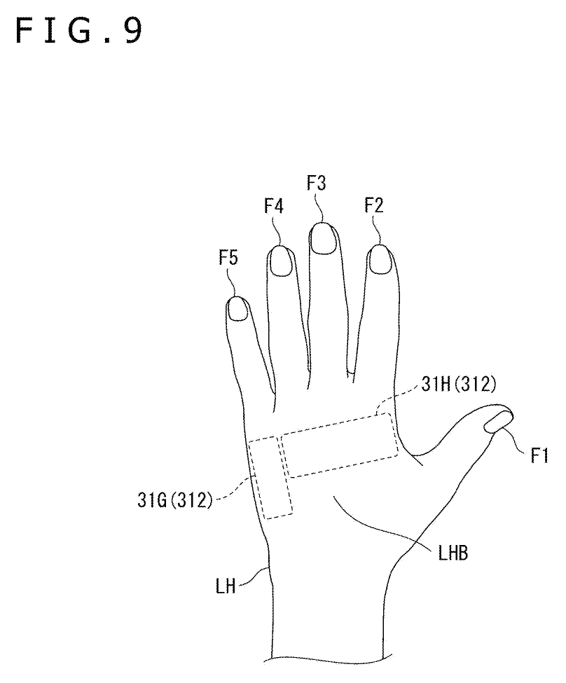

FIG. 9 is a schematic view illustrating an example of the layout of pressing sections 31 on the back LHB of the left hand LH.

In the example of FIG. 9, two pressing sections 31 are provided to the second arrangement section 27.

Each of the two pressing sections 31G and 31H has a rectangular expandable/contractible section 312. As is the case with the example of FIG. 8, the pressing section 31G is provided at a position on the back LHB corresponding to a range from the root of the little finger F5 toward the wrist.

The pressing section 31H is provided at a position corresponding to a substantially central area of the back LHB along the direction in which the fingers F2 through F5 are arrayed.

Specifically, the pressing section 31H is arranged at a position in the substantially central area of the back LHB corresponding to a range from the index finger F2 to the ring finger F4. The expandable/contractible section 312 of the pressing section 31H is substantially the same as the expandable/contractible section 312 of the pressing section 31C in longitudinal dimension. On the other hand, the expandable/contractible section 312 of the pressing section 31H is larger than the expandable/contractible section 312 of the pressing section 31C in transverse dimension.

With the above layout of the pressing sections 31, when the pressing sections 31G and 31H press the back LHB, the user feels a sensation of the back LHB being touched.

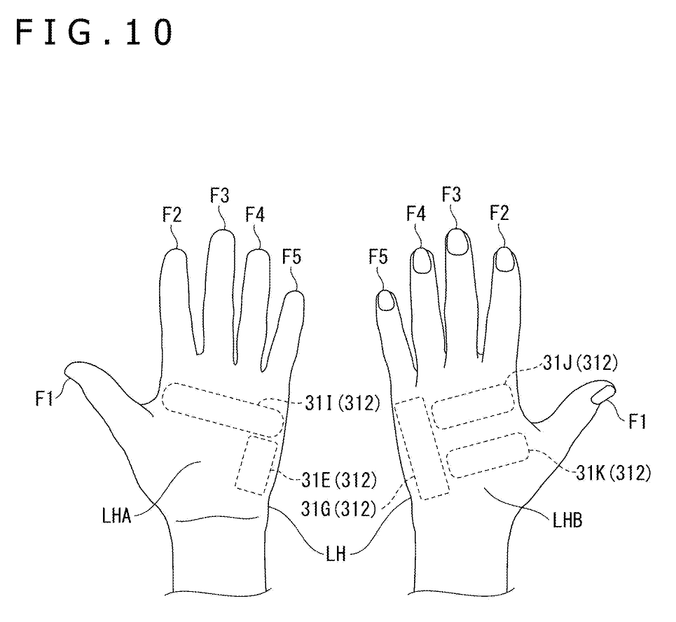

FIG. 10 is a schematic view illustrating an example of the layout of pressing sections 31 on the palm LHA and the back LHB of the left hand LH.

In the example of FIG. 10, two pressing sections 31 are provided to the first arrangement section 26, and three pressing sections 31 are provided to the second arrangement section 27.

Each of the two pressing sections 31I and 31E provided to the first arrangement section 26 has a rectangular expandable/contractible section 312. The pressing section 31I is arranged at a position corresponding to a substantially central area of the palm LHA along the direction in which the fingers F2 through F5 are arrayed. The expandable/contractible section 312 of the pressing section 31I is substantially the same as the expandable/contractible section 312 of the pressing section 31B in transverse dimension. As is the case with the example of FIG. 8, the pressing section 31E is provided at a position on the palm LHA corresponding to the ball of the little finger.

Each of the three pressing sections 31G, 31J, and 31K provided to the second arrangement section 27 has a rectangular expandable/contractible section 312. The pressing sections 31J and 31K are arranged at a position corresponding to a range from the index finger F2 to the ring finger F4 in a substantially central area of the back LHB and arrayed along the direction from the roots of the fingers F2 through F4 toward the wrist. The expandable/contractible section 312 of each of the pressing sections 31J and 31K is smaller than the expandable/contractible section 312 of the pressing section 31I in longitudinal dimension.

The expandable/contractible section 312 of each of the pressing sections 31J and 31K is substantially the same as the expandable/contractible section 312 of the pressing section 31I in transverse dimension. Note that, as is the case with the examples of FIGS. 8 and 9, the pressing section 31G is provided at a position corresponding to a range from the root of the little finger F5 to the wrist on the back LHB.

With the above layout of the pressing sections 31, when each of the pressing sections 31 presses the left hand LH, the user feels a sensation of the left hand LH being gripped by both hands of another person.

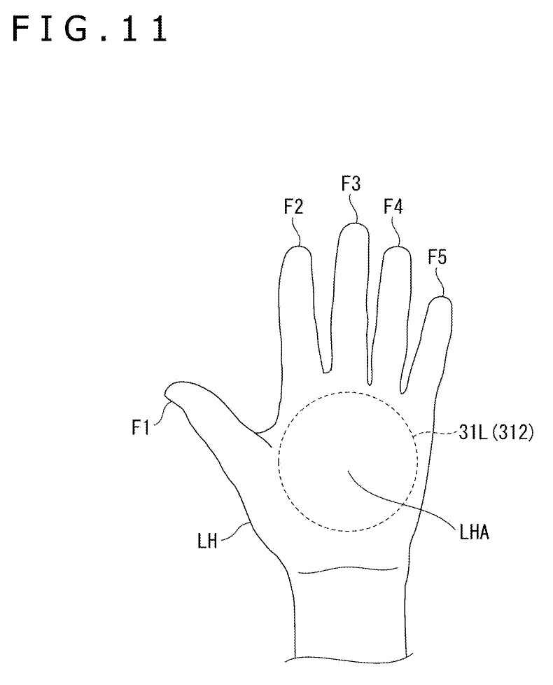

FIG. 11 is a schematic view illustrating an example of the layout of a pressing section 31 on the palm LHA of the left hand LH.

In the example of FIG. 11, one pressing section 31 is provided to the first arrangement section 26.

The pressing section 31 provided to the first arrangement section 26 is referred to as a pressing section 31L provided in a substantially central area of the palm LHA and has an expandable/contractible section 312 that is of a circular shape. The expandable/contractible section 312 of the pressing section 31L has a diameter that is in substantial agreement with the dimension between the root of the index finger F2 and the root of the little finger F5.

With the above layout of the pressing section 31, when the pressing section 31L presses the palm LHA, the user feels a sensation as if a virtual object were created from the palm LHA.

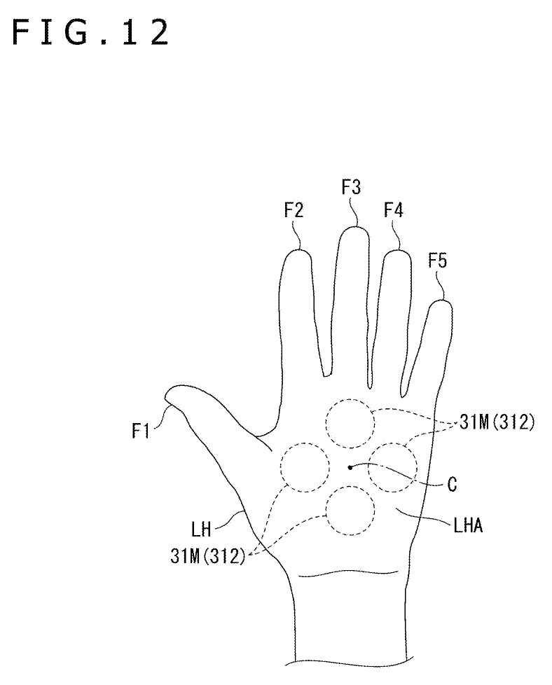

FIG. 12 is a schematic view illustrating an example of the layout of pressing sections 31 on the palm LHA of the left hand LH.

In the example of FIG. 12, four pressing sections 31 are provided to the first arrangement section 26.

Each of the four pressing sections, denoted by 31M, has an expandable/contractible section 312 that is of a circular shape. The four pressing sections 31M are arranged at equal intervals along the circumferential directions around a middle C of the palm LHA. Specifically, of the four pressing sections 31M, one pressing section 31M is arranged closer to the middle finger F3 than the middle C, another pressing section 31M is arranged closer to the wrist than the middle C, still another pressing section 31M is arranged closer to the thumb F1 than the middle C, and the remaining pressing section 31M is arranged across the middle C from the thumb F1.

With the above layout of the pressing sections 31, when the four pressing sections 31 successively press the palm LHA clockwise or counterclockwise around the middle C or press the palm LHA at random, the user can feel a sensation as if a small animal jumped on the palm LHA, for example.

Alternatively, with the above layout, when the four pressing sections 31 simultaneously press the palm LHA, the user can feel a sensation as if an animal such as a dog or a cat touched the palm LHA.

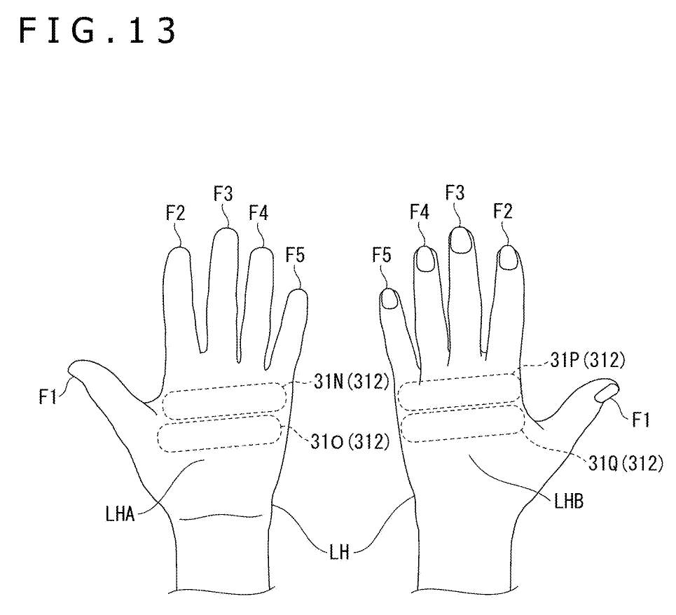

FIG. 13 is a schematic view illustrating an example of the layout of pressing sections 31 on the palm LHA and the back LHB of the left hand LH.

In the example of FIG. 13, two pressing sections 31 are provided to the first arrangement section 26, and two pressing sections 31 are provided to the second arrangement section 27.

Each of the two pressing sections, denoted by 31N and 31O, provided to the first arrangement section 26 has a rectangular expandable/contractible section 312 and is arranged along the direction in which the fingers F2 through F5 are arrayed. Specifically, the pressing section 31N is provided at a position ranging from the root of the little finger F5 to the thumb F1, whereas the pressing section 31O that is arranged closer to the wrist of the left hand LH than the pressing section 31N extends substantially parallel to the pressing section 31N. The longitudinal dimension of the expandable/contractible sections 312 of the pressing sections 31N and 31O is substantially equal to the length of a range from the root of the index finger F2 to the root of the little finger F5.

Each of the two pressing sections, denoted by 31P and 31Q, provided to the second arrangement section 27 has a rectangular expandable/contractible section 312 and is arranged along the direction in which the fingers F2 through F5 are arrayed. Specifically, the pressing section 31P is provided at a position ranging from the root of the index finger f2 to the root of the little finger F5, whereas the pressing section 31Q that is arranged closer to the wrist of the left hand LH than the pressing section 31P extends substantially parallel to the pressing section 31P. The longitudinal dimension of the expandable/contractible sections 312 of the pressing sections 31P and 31Q is substantially equal to the length of a range from the root of the index finger F2 to the root of the little finger F5.

The pressing sections 31N, 31O, 31P, and 31Q are thus arranged such that a hypothetical line successively connecting the pressing section 31P, the pressing section 31N, the pressing section 31Q, and the pressing section 31O follows a helical pattern.

With the above layout of the pressing sections 31, when the pressing section 31P, the pressing section 31N, the pressing section 31Q, and the pressing section 31O successively press the left hand LH or the pressing sections 31N, 31O, 31P, and 31Q simultaneously press the left hand LH, the user can feel a sensation as if a snake were coiled around the left hand LH.

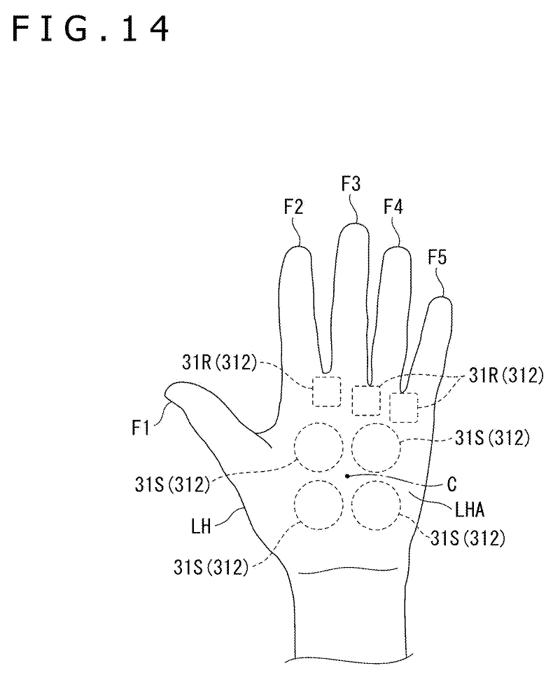

FIG. 14 is a schematic view illustrating an example of the layout of pressing sections 31 on the palm LHA of the left hand LH.

In the example of FIG. 14, seven pressing sections 31 are provided to the first arrangement section 26.

Of the seven pressing sections 31, three pressing sections, denoted by 31R, each have a rectangular expandable/contractible section 312, and four pressing sections, denoted by 31S, each have a circular expandable/contractible section 312.

The three pressing sections 31R are arranged at positions between the roots of the fingers F2 through F5. Specifically, one of the pressing sections 31R is arranged at a position between the root of the index finger F2 and the root of the middle finger F3, another one of the pressing sections 31R is arranged at a position between the root of the middle finger F3 and the root of the ring finger F4, and the remaining one of the pressing sections 31R is arranged at a position between the root of the ring finger F4 and the root of the little finger F5.

The four pressing sections 31S are arranged at equal intervals along the circumferential directions around the middle C of the palm LHA. Specifically, of the four pressing sections 31S, two pressing sections 31S are arranged at positions closer to the fingers F2 through F5 than the middle C along the direction in which the fingers F2 through F5 are arrayed, and the remaining pressing sections 31S are arranged at positions closer to the wrist than the middle C along the direction in which the fingers F2 through F5 are arrayed.

With the above layout of the pressing sections 31, when the pressing sections 31 press the palm LHA, the user can feel a sensation as if sand were falling off the palm LHA.

### Advantages of the first implementation

The pressure sense presentation apparatus 1 according to the present implementation described above offers the following advantages.

The pressure sense presentation apparatus 1 includes the attachment tool 2 and the pressing section 31 that is provided to the attachment tool 2 and presses the attachment target portion to which the attachment tool 2 is attached. According to the present implementation, the pressing section 31 is provided to the strip 21 of the attachment tool 2 and presses the left LH hand as the attachment target portion.

The attachment tool 2 has the strip 21, the through-hole 25, the slit 28, and the locking section 29.

The strip 21 has the first surface 211 and the second surface 212 on the side opposite to the first surface 211 and extends in the +X direction. The +X direction corresponds to the first direction.

The through-hole 25 is provided to the end of the strip 21 in the -X direction and penetrates the strip 21. The -X direction corresponds to the direction opposite to the first direction.

The slit 28 is provided to the strip 21 and spaced from the through-hole 25 in the +X direction. The slit 28 extends along the +X direction and penetrates the strip 21.

The locking section 29 is provided to one surface of the first surface 211 and the second surface 212, contacts the other surface of the first surface 211 and the second surface 212, and locks the other surface. According to the present implementation, the locking section 29 is provided to the second surface 212, contacts the first surface 211, and locks the first surface 211.

With the above configuration, as illustrated in FIGS. 3 through 6, the thumb F1 of the left hand LH is inserted through the through-hole 25, and the strip 21 is wound around the left hand LH, after which the thumb F1 is inserted through the slit 28, and the locking section 29 locks the first surface 211 of the strip 21, whereupon the attachment tool 2 is attached to the left hand LH.

At this time, since the slit 28 extends in the +X direction, the thumb F1 can be inserted through the slit 28 extending in the +X direction, regardless of the lateral dimension of the left hand LH and the thickness of the left hand LH, and the strip 21 wound around the palm LHA and the back LHB of the left hand LH can be locked by the locking section 29.

Consequently, as there is no need to prepare an attachment tool 2 depending on the size of the left hand LH, the attachment tool 2 according to the specification can be versatile in use.

Accordingly, the pressure sense presentation apparatus 1 can also be highly versatile in use regardless of the size of the left hand LH as the attachment target portion to which the attachment tool 2 is to be attached.

In the attachment tool 2, the strip 21 has the first portion 22, the second portion 23, and the third portion 24. The first portion 22 is provided with the through-hole 25. The second portion 23 is positioned in the +X direction with respect to the first portion 22. The third portion 24 is positioned in the +X direction with respect to the second portion 23 and provided with the slit 28. The first portion 22 has expandability/contractibility higher than expandability/contractibility of the second portion 23 and the third portion 24.

With the above configuration, since the through-hole 25 is increased in diameter to an extent depending on the thickness of the thumb F1 inserted through the through-hole 25, the thumb F1 can easily be inserted through the through-hole 25. Moreover, when the attachment tool 2 is attached to the left hand LH, since the first portion 22 is expanded or contracted depending on how the left hand LH moves, the attachment tool 2 is stably attached to the left hand LH.

In the attachment tool 2, the locking section 29 is provided at a position that is on the second surface 232 of the second portion 23 included in the second surface 212 of the strip 21 and that is on the side of the first portion 22.

Here, in a case where the locking section 29 is provided at a position remote from the first portion 22, if the left hand LH is relatively large, the end of the strip 21 opposite to the first portion 22 may fall short of the locking section 29, making it difficult to bring the locking section 29 into engagement with the first surface 211 of the strip 21 in some cases.

By contrast, since the locking section 29 is provided at the position referred to above, it is easy to bring the locking section 29 into engagement with the first surface 211 of the strip 21. Therefore, it is easy to attach the attachment tool 2 to the left hand LH.

In the attachment tool 2, the through-hole 25 is positioned in the +Y direction orthogonal to the +X direction, with respect to the centerline CL that crosses the middle on the first surface 211 in the +Y direction and that lies along the +X direction. The slit 28 is positioned in the -Y direction with respect to the centerline CL. The +Y direction corresponds to the second direction orthogonal to the first direction on the first surface 211. The -Y direction corresponds to a direction opposite to the second direction.

With the above configuration, the dimension of a portion of the strip 21 that extends from the through-hole 25 in the +Y direction is smaller than the dimension of a portion of the strip 21 that extends from the through-hole 25 in the -Y direction. Further, the dimension of a portion of the strip 21 that extends from the slit 28 in the +Y direction is larger than the dimension of a portion of the strip 21 that extends from the through-hole 25 in the -Y direction. In other words, the through-hole 25 and the slit 28 are arranged opposite to each other across the centerline CL.

By contrast, in the left hand LH, the distance from the thumb F1 to the root of the index finger F2 is typically larger than the distance from the thumb F1 to the wrist.

Therefore, when the attachment tool 2 is attached to the left hand LH, the attachment tool 2 can easily be wound around the left hand LH along the direction in which the strip 21 extends. Consequently, the attachment tool 2 can easily be attached to the left hand LH.

In the pressure sense presentation apparatus 1, the strip 21 of the attachment tool 2 has the first arrangement section 26 and the second arrangement section 27. The first arrangement section 26 is provided to the first surface 231 of the second portion 23 included in the first surface 211 of the strip 21, and the pressing section 31 is removably arranged on the first arrangement section 26. The second arrangement section 27 is provided to the first surface 241 of the third portion 24 included in the first surface 211 of the strip 21, and the pressing section 31 is removably arranged on the second arrangement section 27. The first arrangement section 26 and the second arrangement section 27 correspond to an arrangement section.

With the above configuration, the kinds of the pressing sections 31 provided to the arrangement sections 26 and 27 can be changed. Therefore, inasmuch as the pressing sections 31 mounted on the attachment tool 2 can be changed depending on their applications and the like, the pressure sense presentation apparatus 1 can be highly versatile in use.

In the pressure sense presentation apparatus 1, the pressing section 31 has the connecting section 313 that is provided to a surface facing the arrangement section 26 or 27 and that is allowed to be removed from the arrangement section 26 or 27.

With the above configuration, the position where the pressing section 31 is arranged on the arrangement section 26 or 27 can easily be changed depending on its application and the like. Therefore, the pressure sense presentation apparatus 1 can be highly versatile in use.

In the pressure sense presentation apparatus 1, the connecting section 313 and the arrangement section 26 or 27 include respective hook-and-loop fasteners capable of engaging with each other.

With the above configuration, the connecting sections 313 and the arrangement sections 26 and 27 that make the pressing sections 31 removable can be constructed with ease. Therefore, the pressure sense presentation apparatus 1 can be constructed with ease.

The pressure sense presentation apparatus 1 includes the temperature adjusting apparatus 4A that imparts at least one of a warm sensation and a cold sensation to the left hand LH as the attachment target portion.

The above configuration makes it possible to increase the versatility of the pressure sense presentation apparatus 1.

In the pressure sense presentation apparatus 1, the temperature adjusting apparatus 4A is provided to the expandable/contractible section 312 of the pressing section 31. The expandable/contractible section 312 corresponds to a portion for pressing the attachment target portion.

With the above configuration, while the pressing section 31 is contacting the left hand LH, the temperature adjusting apparatus 4A is able to impart at least one of a warm sensation and a cold sensation to the left hand LH.

The pressure sense presentation apparatus 1 includes the vibration generating apparatus 5 that imparts a vibration to the left hand LH as the attachment target portion.

The above configuration increases the versatility of the vibration generating apparatus 5.

In the pressure sense presentation apparatus 1, the vibration generating apparatus 5 is provided to the pressing section 31.

With the above configuration, since a vibration is imparted to the left hand LH that is pressed by the pressing section 31, a person to which the pressure sense presentation apparatus 1 is attached can feel a sensation as if it had contacted a virtual body, for example.

### Second implementation

A second implementation of the present specification will be described below.

A pressure sense presentation apparatus according to the present implementation has a configuration similar to that of the pressure sense presentation apparatus 1 according to the first implementation, but is different therefrom in that a temperature adjusting apparatus is provided to a tube. Note that, in the description below, those parts that are identical or substantially identical to those already described will be denoted by identical reference signs and will be omitted from description.

### Configuration of the pressure sense presentation apparatus

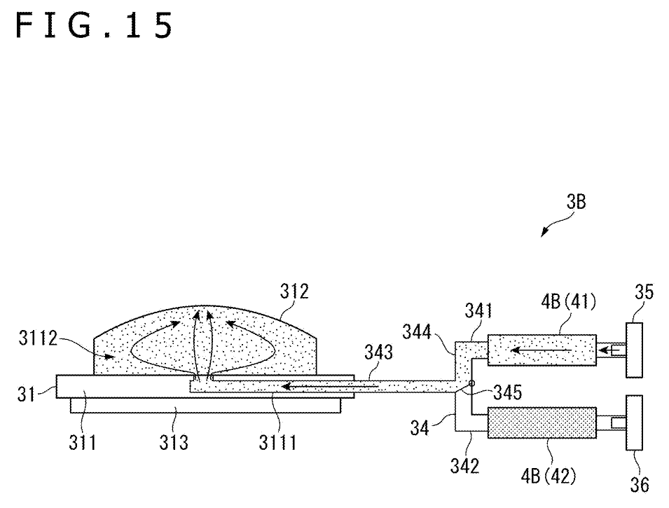

FIG. 15 is a schematic view illustrating a pressing apparatus 3B and a temperature adjusting apparatus 4B of the pressure sense presentation apparatus according to the present implementation.

The pressure sense presentation apparatus according to the present implementation has a configuration and functions similar to those of the pressure sense presentation apparatus 1 according to the first implementation, except that it includes the pressing apparatus 3B and the temperature adjusting apparatus 4B illustrated in FIG. 15 in place of the pressing apparatus 3A and the temperature adjusting apparatus 4A. In other words, the pressure sense presentation apparatus according to the present implementation includes the attachment tool 2, the pressing apparatus 3B, the temperature adjusting apparatus 4B, and the vibration generating apparatus 5.

### Configuration of the pressing apparatus

The pressing apparatus 3B has a configuration similar to that of the pressing apparatus 3A according to the first implementation except that it includes a tube 34, a first pump 35, and a second pump 36 in place of the tube 32 and the pump 33. In other words, the pressing apparatus 3B includes the pressing section 31, the tube 34, the first pump 35, and the second pump 36.

The first pump 35 and the second pump 36 each pump the fluid through the tube 34 into the base 311 of the pressing section 31. The fluid pumped from each of the pumps 35 and 36 may be either gas or liquid.

The tube 34 connects the base 311 of the pressing section 31 to the first pump 35 and the second pump 36. As with the tube 32, the tube 34 represents a distribution pipe through which the fluid flows. The tube 34 has a first inlet portion 341, a second inlet portion 342, a base joint portion 343, a collecting portion 344, and a movable valve 345.

The first inlet portion 341 is connected to the first pump 35. The fluid pumped from the first pump 35 flows into the first inlet portion 341.

The second inlet portion 342 is connected to the second pump 36. The fluid pumped from the second pump 36 flows into the second inlet portion 342.

The base joint portion 343 is connected to the base 311.

The collecting portion 344 is connected to the first inlet portion 341, the second inlet portion 342, and the base joint portion 343.

The movable valve 345 is provided in the collecting portion 344. The movable valve 345 switches between a fluid passage through which the fluid flowing in from the first inlet portion 341 is introduced into the base joint portion 343 and a second passage through which the fluid flowing in from the second inlet portion 342 is introduced into the base joint portion 343. The movable valve 345 described above may be arranged such that its fluid passage switching operation can be controlled by an undepicted controlling section of the pressure sense presentation apparatus, or may be arranged such that its fluid passage switching operation can automatically be performed by the pressure of the fluid pumped from the pumps 35 and 36.

### Configuration of the temperature adjusting apparatus

As with temperature adjusting apparatus 4A according to the first implementation, the temperature adjusting apparatus 4B imparts at least one of a warm sensation and a cold sensation to the attachment target portion to which the attachment tool 2 is attached. The temperature adjusting apparatus 4B has a first temperature adjuster 41 and a second temperature adjuster 42.

The first temperature adjuster 41 is provided in the first inlet portion 341 of the tube 34 as the distribution pipe. The first temperature adjuster 41 heats the fluid pumped from the first pump 35. Such a first temperature adjuster 41 may be of a configuration having a Peltier device or a nichrome wire by way of example.

The second temperature adjuster 42 is provided in the second inlet portion 342 of the tube 34 as the distribution pipe. The second temperature adjuster 42 cools the fluid pumped from the second pump 36. Such a second temperature adjuster 42 may be of a configuration having a Peltier device by way of example.

### Operation of the pressing apparatus and the temperature adjusting apparatus

For imparting a warm sensation to the attachment target portion, the first pump 35 and the first temperature adjuster 41 function. In this case, the fluid pumped from the first pump 35 is heated by the first temperature adjuster 41, so that the fluid at a higher temperature flows through the tube 34 into the inlet chamber 3112 in the base 311. Since such fluid increases the temperature of the expandable/contractible section 312 held in contact with the attachment target portion, it imparts a warm sensation to the attachment target portion.

For imparting a cold sensation to the attachment target portion, the second pump 36 and the second temperature adjuster 44 function. In this case, the fluid pumped from the second pump 36 is cooled by the second temperature adjuster 42, so that the fluid at a lower temperature flows through the tube 34 into the inlet chamber 3112 in the base 311. Since such fluid reduces the temperature of the expandable/contractible section 312 held in contact with the attachment target portion, it imparts a cool sensation to the attachment target portion.

Note that, if a warm sensation and a cold sensation are not to be imparted, then the temperature adjusting apparatus 4B may not heat and cool the fluid. In this case, the fluid may be pumped by either the first pump 35 or the second pump 36.

### Advantages of the second implementation

The pressure sense presentation apparatus according to the present implementation described above offers the following advantages as well as advantages similar to those of the pressure sense presentation apparatus 1 according to the first implementation.

In the pressure sense presentation apparatus according to the present implementation, the pressing section 31 has the base 311 having the inlet chamber 3112 where the fluid flows in and the expandable/contractible section 312 that is provided to the base 311, is caused to expand and contract by the fluid having flowed into the inlet chamber 3112, and presses the left hand LH. The left hand LH represents the attachment target portion of the user to which the attachment tool 2 is attached, and the temperature adjusting apparatus 4B adjusts the temperature of the fluid.

With the above configuration, the pressing section 31 can press the left hand LH and impart a warm sensation and a cold sensation to the left hand LH at the same time. Further, inasmuch as the temperature adjusting apparatus 4B adjusts the temperature of the fluid to be supplied to the inlet chamber 3112 in the pressing section 31, the weight of a constituent element of the pressure sense presentation apparatus that is attached to the attachment target portion can be prevented from becoming larger than if the temperature adjusting apparatus is provided in the pressing section.

The pressure sense presentation apparatus according to the present implementation includes the pumps 35 and 36 that pump the fluid and the tube 34 that is provided between the base 311 and the pumps 35 and 36 and distributes the fluid pumped from the pumps 35 and 36 to the base. The tube 34 corresponds to a distribution pipe.

The temperature adjusting apparatus 4B is provided to the tube 34. Specifically, the first temperature adjuster 41 of the temperature adjusting apparatus 4B is provided to the first inlet portion 341 of the tube 34, and the second temperature adjuster 42 is provided to the second inlet portion 342 of the tube 34.

With the above configuration, the temperature adjusting apparatus 4B is able to adjust the temperature of the fluid supplied from the pumps 35 and 36 to the pressing section 31. Therefore, a warm sensation and a cold sensation can be imparted to the left hand LH.

### Third implementation

A third implementation of the present specification will be described below.

A pressure sense presentation apparatus according to the present implementation has a configuration similar to that of the pressure sense presentation apparatus 1 according to the first implementation, but is different therefrom as to the configuration of the temperature adjusting apparatus. Note that, in the description below, those parts that are identical or substantially identical to those already described will be denoted by identical reference signs and will be omitted from description.

### Configuration of the pressure sense presentation apparatus

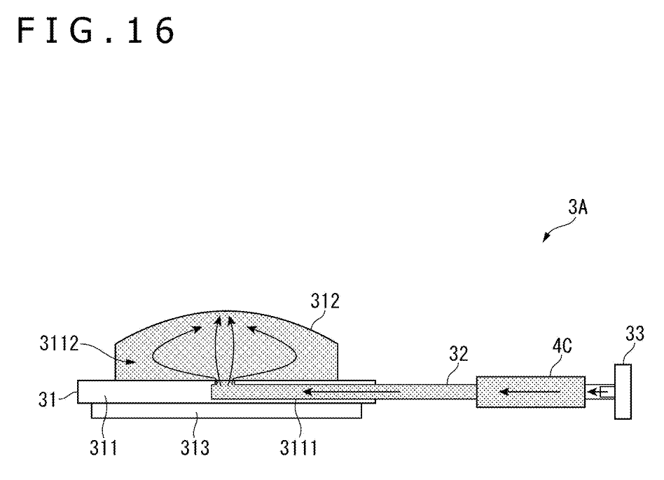

FIG. 16 is a schematic view illustrating a pressing apparatus 3A and a temperature adjusting apparatus 4C of the pressure sense presentation apparatus according to the present implementation.

The pressure sense presentation apparatus according to the present implementation has a configuration and functions similar to those of the pressure sense presentation apparatus 1 according to the first implementation, except that it includes the temperature adjusting apparatus 4C illustrated in FIG. 16 in place of the temperature adjusting apparatus 4A. In other words, the pressure sense presentation apparatus according to the present implementation includes the attachment tool 2, the pressing apparatus 3A, the temperature adjusting apparatus 4C, and the vibration generating apparatus 5.

### Configuration of the temperature adjusting apparatus

As with the temperature adjusting apparatus 4A according to the first implementation and the temperature adjusting apparatus 4B according to the second implementation, the temperature adjusting apparatus 4C imparts at least one of a warm sensation and a cold sensation to the attachment target portion to which the attachment tool 2 is attached.

The temperature adjusting apparatus 4C is provided to the tube 32 that represents a distribution pipe of the pressing apparatus 3A. The temperature adjusting apparatus 4C heats or cools the fluid that is pumped from the pump 33 and that flows through the tube 32, to adjust the temperature of the fluid. Such a temperature adjusting apparatus 4C may be of a configuration having a Peltier device capable of both heating and cooling the fluid by way of example. Note that the temperature adjusting apparatus 4C may alternatively have a heater for heating the fluid and a cooler for cooling the fluid, the heater including a nichrome wire, for example, and the cooler including a Peltier device, for example. In this case, the heater and the cooler may individually be disposed in the tube 32.

### Operation of the temperature adjusting apparatus

For imparting a warm sensation to the attachment target portion, the temperature adjusting apparatus 4C heats the fluid pumped from the pump 33, and the fluid at a higher temperature flows through the tube 32 into the inlet chamber 3112 in the base 311. Since such fluid increases the temperature of the expandable/contractible section 312 held in contact with the attachment target portion, it imparts a warm sensation to the attachment target portion.

For imparting a cold sensation to the attachment target portion, the temperature adjusting apparatus 4C cools the fluid pumped from the pump 33, and the fluid at a lower temperature flows through the tube 32 into the inlet chamber 3112 in the base 311. Since such fluid reduces the temperature of the expandable/contractible section 312 held in contact with the attachment target portion, it imparts a cool sensation to the attachment target portion.

Note that, for selectively imparting the warm sensation, imparting the cold sensation, and not imparting the warm sensation and the cold sensation at short intervals of time, the pump 33 may positively draw in the fluid from the inlet chamber 3112.

### Advantages of the third implementation

The pressure sense presentation apparatus according to the present implementation described above offers advantages similar to those of the pressure sense presentation apparatus 1 according to the first implementation and the pressure sense presentation apparatus according to the second implementation.

### Modifications of the implementations

The present specification is not limited to the above implementations, and modifications and improvements that fall within the scope for achieving the object of the present specification are covered by the present specification.

According to each of the above implementations, the attachment tool 2 has been described as being attached to the left hand LH that is one of attachment target portions. However, the attachment tool is not limited to use on the left hand, and the attachment tool according to the present specification may be attached to the right hand RH. In the latter case, the attachment tool may be a mirror image of the attachment tool 2 for the left hand LH illustrated in FIG. 1. In addition, the finger to be inserted through the through-hole 25 of the attachment tool 2 is not limited to the thumb, and may be other fingers including the index finger and the little finger, for example. In the latter case, the attachment tool 2 may be wound around one hand in a manner to extend toward the first metacarpal bone, the fifth metacarpal bone, or the first interdigital space between the thumb and the index finger.

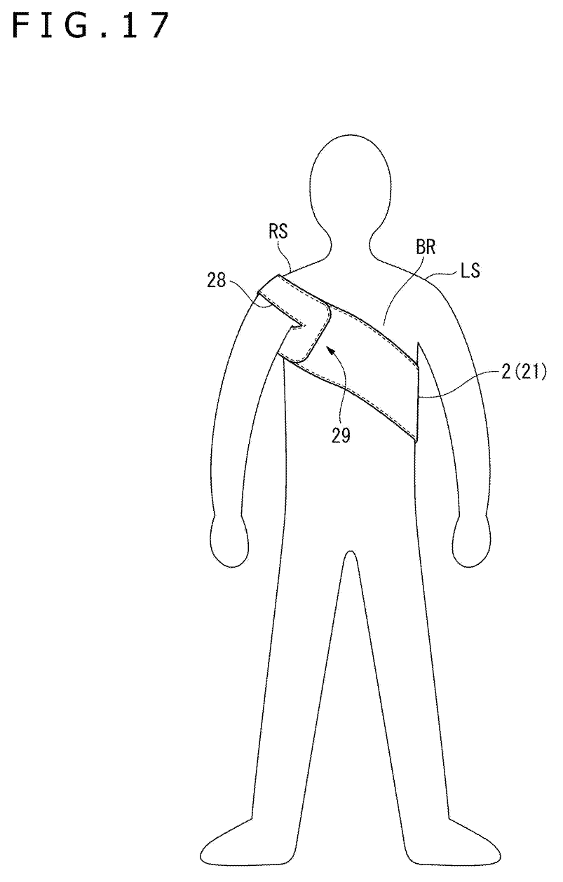

FIG. 17 is a schematic view illustrating another example of attaching the attachment tool 2.

The attachment tool 2 may alternatively be attached to other parts of the body. For example, as illustrated in FIG. 17, the attachment tool 2 may be attached to a part of the body by being wound over a right shoulder RS, a chest BR, and the back. Specifically, the attachment tool 2 can be attached to an upper part of the body by inserting the right shoulder RS through the through-hole 25 of the attachment tool 2, placing the first surface 211 of the strip 21 along the chest BR and the back, then inserting the right shoulder RS through the slit 28, and locking the first surface 211 with the locking section 29. The attachment tool 2 may similarly be attached to a part of the body by being wound over a left shoulder LS, the chest BR, and the back. Further, the same applies to a case of attaching the attachment tool 2 to a part of the body by winding it over the joint of a leg and the right shoulder RS or the left shoulder LS and a case of attaching the attachment tool 2 to a part of the body by winding it over the head and an armpit or groin. For attaching the attachment tool 2 to a part of the body by winding it over the joint of a leg and the right shoulder RS or the left shoulder LS, the leg may be inserted through the through-hole 25 and the slit 28. For attaching the attachment tool 2 to a part of the body by winding it over the head and an armpit or groin, the head may be inserted through the through-hole 25 and the slit 28.

Moreover, the attachment tool 2 may be attached to a foot more distal to the ankle. For example, the attachment tool 2 with a toe inserted through the through-hole 25 may be wound around the foot in a manner to extend toward the first metatarsal bone or the fifth metatarsal bone. In this case, the attachment tool 2 can be attached to the leg by locking the first surface 211 with the locking section 29 after the toe has been inserted through the slit 28.

According to each of the above implementations, the locking section 29 is provided to the second surface 212 of the strip 21 and locks the first surface 211 of the strip 21. However, the locking section 29 is not limited to such details, and may be provided to the first surface 211 of the strip 21 and lock the second surface 212 of the strip 21. In the latter case, the locking section 29 may be provided to the end of the first surface 211 that is opposite to the first portion 22.

According to each of the above implementations, the expandability/contractibility of the first portion 22 is higher than expandability/contractibility of the second portion 23 and the expandability/contractibility of the third portion 24. However, the expandability/contractibility of the first portion 22 is not limited to such a feature. As long as the first portion 22 with the through-hole 25 provided therein is expandable/contractible, the expandability/contractibility of the first portion 22 may be equal to or lower than the expandability/contractibility of each of the second portion 23 and the third portion 24.

Moreover, the slit 28 is provided to the third portion 24. However, the slit 28 is not limited to such a feature, and may be provided to both the second portion 23 and the third portion 24.

According to each of the above implementations, the strip 21 has the first surface 211 as a surface facing the attachment target portion and the second surface 212 on the side opposite to the first surface 211, and the locking section 29 is provided at a position that is on the second surface 232 of the second portion 23 included in the second surface 212 and that is on the side of the first portion 22. However, the strip 21 is not limited to such details. The position of the locking section 29 may be changed appropriately. For example, the locking section 29 may be provided at a position that is on the second surface 232 and that is on the side opposite to the first portion 22 or may be provided at a position that is on the second surface 242 of the third portion 24 and that is on the side of the first portion 22.

According to each of the above implementations, the through-hole 25 is provided at a position spaced from the centerline CL in the +Y direction, and the slit 28 is provided at a position spaced from the centerline CL in the -Y direction. However, the through-hole 25 and the slit 28 are not limited to such details. The position of the through-hole 25 and the position of the slit 28 on the strip 21 may be changed appropriately. For example, the through-hole 25 may be provided at a position spaced from the centerline CL in the -Y direction, and the slit 28 may be provided at a position spaced from the centerline CL in the +Y direction. Moreover, for example, at least one of the through-hole 25 and the slit 28 may be provided on the centerline CL. Further, for example, the through-hole 25 and the slit 28 may each be shifted from the centerline CL in one of the +Y direction and the -Y direction.

According to each of the above implementations, the pressure sense presentation apparatus includes the pressing apparatus 3A or 3B, the temperature adjusting apparatus 4A, 4B, or 4C, and the vibration generating apparatus 5. However, the pressure sense presentation apparatus is not limited to such details. The pressure sense presentation apparatus may be free of at least one of the pressing apparatus, the temperature adjusting apparatus, and the vibration generating apparatus. For example, the pressure sense presentation apparatus may not necessarily include the temperature adjusting apparatus and the vibration generating apparatus.

According to each of the above implementations, the pressing section 31 has connecting section 313 that can be connected to one of the first arrangement section 26 and the second arrangement section 27, and the connecting section 313, the first arrangement section 26, and the second arrangement section 27 each include a hook-and-loop fastener. However, the present specification is not limited to such details. The first arrangement section 26 and the second arrangement section 27 may be provided with a holding structure that holds the base 311 of the pressing section 31. In other words, the pressing section 31 may be mounted on the strip 21 by another structure.

According to each of the above implementations, in the pressing apparatus 3A or 3B, the expandable/contractible section 312 that can be expanded and contracted by the fluid pumped from the pump 33 or the pumps 35 and 36 presses the attachment target portion to which the attachment tool 2 is attached. However, the pressing apparatus is not limited such a configuration. For example, the pressing apparatus may be caused to protrude to press the attachment target portion with a drive device such as an electric motor.

According to the above implementations, the temperature adjusting apparatuses 4A, 4B, and 4C are arranged to be able to impart each of the warm sensation and the cold sensation to the attachment target portion. However, the temperature adjusting apparatuses 4A, 4B, and 4C are not limited to such features. The temperature adjusting apparatuses 4A, 4B, and 4C may impart one of the warm sensation and the cold sensation.

According to each of the above implementations, the vibration generating apparatus 5 is provided on the base 311 of the pressing section 31. However, the vibration generating apparatus 5 is not limited to such a feature, and may be provided on the attachment tool 2 separately from the pressing section 31. Similarly, in the pressure sense presentation apparatus 1 according to the first implementation, the temperature adjusting apparatus 4A may also be provided on the attachment tool 2 separately from the pressing section 31.

According to each of the above implementations, the attachment tool 2 constitutes the pressure sense presentation apparatus. For example, the attachment tool 2 supports the pressing section 31 of the pressing apparatus 3A or 3B, the temperature adjusting apparatus 4A, and the vibration generating apparatus 5. However, the attachment tool is not limited to such features. The attachment tool according to the present specification may be used as an attachment tool for attaching other apparatuses to a body or an object. For example, the attachment tool according to the present specification may be used to attach an image capturing device such as a camera and various sensors for detecting motion of a body or an object to the body or the object. Moreover, for example, the attachment tool according to the present specification may be used as medical supplies such as a supporter and as part of clothes and bags.

Further, the attachment tool according to the present specification may not necessarily be attached to a body, and may be attached to an object.

### Overview of the present specification

An overview of the present specification will be added below.

1 An attachment tool including:

a strip that has a first surface and a second surface on a side opposite to the first surface and extends in a first direction;

a through-hole that is provided to an end of the strip positioned in a direction opposite to the first direction and penetrates the strip;

a slit that is provided to the strip in the first direction with respect to the through-hole, extends along the first direction, and penetrates the strip; and

a locking section that is provided to one surface of the first surface and the second surface, contacts the other surface of the first surface and the second surface, and locks the other surface.

With the above configuration, the attachment tool can be attached to a body or an object by inserting part of the body or part of the object, such as a thumb or an arm, through the through-hole, winding the strip around the body or the object, then threading the part through the slit, and locking the first surface and the second surface of the strip with the locking section. For example, in a case where the attachment tool is to be attached to the left hand, the attachment tool can be attached to the left hand by inserting the thumb of the left hand through the through-hole, winding the strip around the palm and back of the left hand, then threading the thumb through the slit, and locking the strip with the locking section.

Since the slit extends along the first direction at this time, the strip can be wound around the body or the object regardless of the size of the attachment target portion to which the attachment tool is to be attached. For example, in a case where the attachment tool is to be attached to the left hand, regardless of the lateral dimension of the left hand and the thickness of the left hand, the thumb of the left hand is threaded through the slit that extends in the first direction, and the strip wound around the palm and back of the left hand can be locked by the locking section. Consequently, as there is no need to prepare an attachment tool depending on the size of the attachment target portion, the attachment tool can be versatile in use.

2 The attachment tool according to [1], in which

the strip has

a first portion provided with the through-hole,

a second portion positioned in the first direction with respect to the first portion, and

a third portion positioned in the first direction with respect to the second portion and provided with the slit, and

the first portion has expandability/contractibility higher than expandability/contractibility of the second portion and the third portion.

With the above configuration, since the expandability/contractibility of the first portion where the through-hole is provided is higher than the expandability/contractibility of the other portions, the through-hole is increased in diameter to an extent depending on the thickness of the part of the body or the part of the object that is inserted through the through-hole. Thus, the part of the body or the part of the object can easily be inserted through the through-hole. Further, when the attachment tool is attached to the body or the object, as the first portion can be expanded or contracted depending on how the body or the object moves, the attachment tool is stably attached to the body.

3 The attachment tool according to [2], in which

the locking section is provided at a position that is on a surface of the second portion included in the second surface and that is on a side of the first portion.

Here, in a case where the locking section is provided at a position remote from the first portion, if the part of the body to which the attachment tool is attached is relatively large, the end of the strip opposite to the first portion may fall short of the locking section, making it difficult to bring the locking section into engagement with the first surface of the strip in some cases.

By contrast, since the locking section is provided at the position referred to above, it is easy to bring the locking section into engagement with the first surface of the strip. Therefore, it is easy to attach the attachment tool to the body.

4 The attachment tool according to any one of [1] through [3], in which

the through-hole is positioned in a second direction orthogonal to the first direction, with respect to a centerline that crosses a middle on the first surface in the second direction and that lies along the first direction, and

the slit is positioned in a direction opposite to the second direction, with respect to the centerline.

With the above configuration, the dimension of a portion of the strip that extends from the through-hole in the second direction is smaller than the dimension of a portion of the strip that extends from the through-hole in the direction opposite to the second direction. Further, the dimension of a portion of the strip that extends from the slit in the second direction is larger than the dimension of a portion of the strip that extends from the slit in the direction opposite to the second direction. In other words, the through-hole and the slit are arranged opposite to each other across the centerline.

Consequently, in the attachment target portion of a body to which the attachment tool is to be attached, if the distance of the portion in the direction opposite to the second direction from the part to be inserted through the through-hole is larger than the distance of the portion in the second direction from the part to be inserted through the through-hole, then it is easy to wind the attachment tool around the attachment target portion along the direction in which the strip extends. For example, on the left hand, since the distance from the thumb to the root of the index finger is typically larger than the distance from the thumb to the wrist, when the attachment tool is to be attached to the left hand, the attachment tool can easily be wound around the left hand along the direction in which the strip extends.

Therefore, the attachment tool can easily be attached to the body.

5 A pressure sense presentation apparatus including:

the attachment tool according to [2] or [3]; and

a pressing section that is provided to the strip and presses an attachment target portion to which the attachment tool is attached.

The above configuration offers advantages similar to those of the attachment tool described above. The pressure sense presentation apparatus can be highly versatile in use as it can be attached to the attachment target portion regardless of the size of the attachment target portion.

6 The pressure sense presentation apparatus according to [5], in which

the attachment tool has an arrangement section where the pressing section is arranged removably, the arrangement section being provided to a surface included in the first surface at at least one portion of the second portion and the third portion.

With the above configuration, the kind of the pressing section disposed on the attachment section can be changed. Consequently, since the pressing section mounted on the attachment tool can be changed depending on its application and the like, the pressure sense presentation apparatus can be highly versatile in use.

7 The pressure sense presentation apparatus according to [6], in which

the pressing section has a connecting section that is provided to a surface facing the arrangement section and that is allowed to be removed from the arrangement section.

With the above configuration, since the position where the pressing section is arranged on the arrangement section can easily be changed depending on its application and the like, the pressure sense presentation apparatus can be highly versatile in use.

8 The pressure sense presentation apparatus according to [7], in which

the connecting section and the arrangement section include respective hook-and-loop fasteners capable of engaging with each other.

With the above configuration, the connecting section and the arrangement section that make the pressing section removable can be constructed with ease. Therefore, the pressure sense presentation apparatus can be constructed with ease.

9 The pressure sense presentation apparatus according to any one of [5] through [8], including:

a temperature adjusting apparatus that imparts at least one of a warm sensation and a cold sensation to the attachment target portion.

With the above configuration, the pressure sense presentation apparatus is made higher in versatility.

The pressure sense presentation apparatus according to [9], in which

the temperature adjusting apparatus is provided to a portion of the pressing section where the pressing portion presses the attachment target portion.

With the above configuration, while the pressing section is in contact with the attachment target portion, the temperature adjusting apparatus is able to impart at least one of a warm sensation and a cold sensation to the attachment target portion.

The pressure sense presentation apparatus according to [9], in which

the pressing section has

a base having an inlet chamber where fluid flows in, and

an expandable/contractible section that is provided to the base, is caused to expand and contract by the fluid having flowed into the inlet chamber, and presses the attachment target portion, and

the temperature adjusting apparatus adjusts a temperature of the fluid.

With the above configuration, the pressing section can press the attachment target portion and imparts at least one of a warm sensation and a cold sensation to the attachment target portion at the same time. Further, inasmuch as the temperature adjusting apparatus adjusts the temperature of the fluid to be supplied to the inlet chamber in the pressing section, the weight of a constituent element of the pressure sense presentation apparatus that is attached to the body or the object can be prevented from becoming larger than if the temperature adjusting apparatus is provided to the pressing section.

The pressure sense presentation apparatus according to , including:

a pump that pumps the fluid; and

a distribution pipe that is provided between the base and the pump and distributes the fluid pumped from the pump to the base, in which

the temperature adjusting apparatus is provided to the distribution pipe.

With the above configuration, the temperature adjusting apparatus is able to adjust the temperature of the fluid supplied from the pump to the pressing section.

The pressure sense presentation apparatus according to any one of [5] through , including:

a vibration generating apparatus that imparts a vibration to the attachment target portion.

With the above configuration, the versatility of the pressure sense presentation apparatus can be increased.

The pressure sense presentation apparatus according to , in which

the vibration generating apparatus is provided to the pressing section.

With the above configuration, since a vibration is imparted to the attachment target portion that is pressed by the pressing section, a person to which the pressure sense presentation apparatus is attached can feel a sensation as if it had contacted a virtual body, for example.

### Reference Signs List

1: Pressure sense presentation apparatus

2: Attachment tool

21: Strip

211: First surface

212: Second surface

22: First portion

23: Second portion

231: First surface

232: Second surface

24: Third portion

241: First surface

242: Second surface

25: Through-hole

26: First arrangement section (arrangement section)

27: Second arrangement section (arrangement section)

28: Slit

29: Locking section

3A, 3B: Pressing apparatus

31: Pressing section

311: Base

3111: Fluid passage

3112: Inlet chamber

32, 34: Tube (distribution pipe)

33: Pump

35: First pump

36: Second pump

4A, 4B, 4C: Temperature adjusting apparatus

41: First temperature adjuster

42: Second temperature adjuster

5: Vibration generating apparatus

CL: Centerline

1. An attachment tool comprising: a strip that extends in a first direction and that has (i) a first surface, and (ii) a second surface on a side opposite to the first surface; a through-hole that penetrates the strip and that that is provided at an end of the strip that is opposite to the first direction; a slit that penetrates the strip and that is provided in the first direction with respect to the through-hole; and a locking section that is provided to one of the first surface or the second surface, and that contacts and locks an opposite surface to the one of the first surface or the second surface.

2. The attachment tool of claim 1, wherein: the strip comprises: a first portion that is provided with the through-hole, a second portion that is positioned in the first direction with respect to the first portion, and a third portion that positioned in the first direction with respect to the second portion and that is provided with the slit, and the first portion has an expandability or contractibility that is higher than a corresponding expandability or contractibility of the second portion and the third portion.

3. The attachment tool of claim 2, wherein: the locking section is provided at a position that is on a surface of the second portion included in the second surface and that is on a side of the first portion.

4. The attachment tool of claim 1, wherein: the through-hole is positioned in a second direction that is orthogonal to the first direction, with respect to a centerline that crosses a middle on the first surface in the second direction and that lies along the first direction, and the slit is positioned in a direction that is opposite to the second direction, with respect to the centerline.

5. A pressure sense presentation apparatus comprising: an attachment tool comprising: a strip that extends in a first direction and that has (i) a first surface, and (ii) a second surface on a side opposite to the first surface; a through-hole that penetrates the strip and that that is provided at an end of the strip that is opposite to the first direction; a slit that penetrates the strip and that is provided in the first direction with respect to the through-hole; and a locking section that is provided to one of the first surface or the second surface, and that contacts and locks an opposite surface to the one of the first surface or the second surface, wherein the strip comprises: a first portion that is provided with the through-hole, a second portion that is positioned in the first direction with respect to the first portion, and a third portion that positioned in the first direction with respect to the second portion and that is provided with the slit, and the first portion has an expandability or contractibility that is higher than a corresponding expandability or contractibility of the second portion and the third portion,a pressing section that is provided to the strip and presses an attachment target portion to which the attachment tool is attached.

6. The pressure sense presentation apparatus of claim 5, wherein: the attachment tool has an arrangement section where the pressing section is removably arranged, the arrangement section is provided to a surface included in the first surface in at least one portion of the second portion and the third portion.

7. The pressure sense presentation apparatus of claim 6, wherein: the pressing section has a connecting section that is provided to a surface facing the arrangement section and that is allowed to be removed from the arrangement section.

8. The pressure sense presentation apparatus of claim 7, wherein: the connecting section and the arrangement section include respective hook-and-loop fasteners capable of engaging with each other.

9. The pressure sense presentation apparatus of claim 5, comprising: a temperature adjusting apparatus that imparts at least one of a warm sensation or a cold sensation to the attachment target portion.

10. The pressure sense presentation apparatus of claim 9, wherein: the temperature adjusting apparatus is provided to a portion of the pressing section where the pressing portion presses the attachment target portion.

11. The pressure sense presentation apparatus of claim 9, wherein: the pressing section comprises: a base having an inlet chamber where fluid flows in, andan expandable or contractible section that is provided to the base, and that is configured to expand and contract by the fluid having flowed into the inlet chamber, and and that presses the attachment target portion, and the temperature adjusting apparatus adjusts a temperature of the fluid.

12. The pressure sense presentation apparatus of claim 11, comprising: a pump that is configured to pump the fluid; and a distribution pipe that is provided between the base and the pump, and that is configured to distribute the fluid pumped from the pump to the base, wherein the temperature adjusting apparatus is provided to the distribution pipe.

13. The pressure sense presentation apparatus according to claim 5, comprising: a vibration generating apparatus that is configured to impart a vibration to the attachment target portion.

14. The pressure sense presentation apparatus according to claim 13, wherein the vibration generating apparatus is provided to the pressing section.

15. A method comprising: providing a strip that extends in a first direction and that has (i) a first surface, and (ii) a second surface on a side opposite to the first surface; providing a through-hole that penetrates the strip and that that is provided at an end of the strip that is opposite to the first direction; providing a slit that penetrates the strip and that is provided in the first direction with respect to the through-hole; and providing a locking section that is provided to one of the first surface or the second surface, and that contacts and locks an opposite surface to the one of the first surface or the second surface.

16. The method of claim 15, wherein: the strip comprises: a first portion that is provided with the through-hole, a second portion that is positioned in the first direction with respect to the first portion, and a third portion that positioned in the first direction with respect to the second portion and that is provided with the slit, and the first portion has an expandability or contractibility that is higher than a corresponding expandability or contractibility of the second portion and the third portion.

17. The method of claim 16, wherein: the locking section is provided at a position that is on a surface of the second portion included in the second surface and that is on a side of the first portion.

18. The method claim 1, wherein: the through-hole is positioned in a second direction that is orthogonal to the first direction, with respect to a centerline that crosses a middle on the first surface in the second direction and that lies along the first direction, and the slit is positioned in a direction that is opposite to the second direction, with respect to the centerline.

19. A method comprising: providing an attachment tool comprising: a strip that extends in a first direction and that has (i) a first surface, and (ii) a second surface on a side opposite to the first surface; a through-hole that penetrates the strip and that that is provided at an end of the strip that is opposite to the first direction; a slit that penetrates the strip and that is provided in the first direction with respect to the through-hole; and a locking section that is provided to one of the first surface or the second surface, and that contacts and locks an opposite surface to the one of the first surface or the second surface, wherein the strip comprises: a first portion that is provided with the through-hole, a second portion that is positioned in the first direction with respect to the first portion, and a third portion that positioned in the first direction with respect to the second portion and that is provided with the slit, and the first portion has an expandability or contractibility that is higher than a corresponding expandability or contractibility of the second portion and the third portion, providing a pressing section to the strip and presses an attachment target portion to which the attachment tool is attached.

20. The method of claim 19, wherein: the attachment tool has an arrangement section where the pressing section is removably arranged, the arrangement section is provided to a surface included in the first surface in at least one portion of the second portion and the third portion.
